(19) United States
(12) Patent Application Publication
(10) Pub. No.: US 2025/0288317 A1
(43) Pub. Date: Sep. 18, 2025

(54) PERFUSION BALLOON CATHETER AND RELATED METHODS

(71) Applicant: Bard Peripheral Vascular, Inc., Franklin Lakes, NJ (US)

(72) Inventors: Jessica Johnson, Plano, TX (US); Jennifer Lee, Plano, TX (US); Christian Mendiondo, San Antonio, TX (US); Zachary Mendoza, Keller, TX (US); Faizaan Khan, Tomball, TX (US); Deep Parikh, College Station, TX (US); Alex Palmer, Scottsdale, AZ (US)

(21) Appl. No.: 18/861,463

(22) PCT Filed: Apr. 29, 2022
(86) PCT No.: PCT/US2022/026900

§ 371 Date: Oct. 29, 2024

(51) Int. Cl. A61B 17/34; A61B 17/00; A61M 25/00; A61M 25/10

(52) U.S. Cl. CPC A61B 17/34; A61B 17/00234; A61M 25/0043; A61M 25/1002; A61M 25/1011; A61B 2017/00238; A61B 2017/00305; A61B 2017/00557; A61M 2025/1045; A61M 2025/1086; A61M 2025/1093; A61M 2025/1097; A61M 2210/12

(57) ABSTRACT

An apparatus for performing a medical procedure in a main vessel, includes an inflatable perfusion balloon forming an internal passage for allowing fluid flow within the vessel when the balloon is inflated. A shaft connected to the balloon includes an inflation lumen in communication with the perfusion balloon, and a working lumen including an opening for passing a tool, such as a needle for puncturing the vessel. An indexing balloon may be provided for expanding into a branch vessel to orient the working lumen in the direction of the branch vessel, such as to allow for an incision to be made at a desired location for achieving a by-pass. The perfusion balloon may be adapted for receiving a distal end portion of the shaft, which may include an outlet of the working lumen substantially flush with the external surface. Related methods are also disclosed.

### BACKGROUND OF THE INVENTION

Repair of the vasculature often requires an invasive surgical procedure, which is desirable to avoid or at least minimize whenever possible due to the resulting risks and potential complications. Over the years, various endovascular devices, such as balloon catheters, have been developed to perform various types of vascular repairs without such an invasive procedure. This currently includes devices specially adapted for puncturing the vessel wall to allow for the placement of a bypass device, such as in a transjugular intrahepatic portosystemic shunt (TIPS) procedure, where a stent graft connects the portal veins to adjacent, lower pressure blood vessels.

Known apparatuses that facilitate puncturing for such a bypass procedure typically does not provide a means for isolating the location of the puncture site from blood flow during the procedure. This can add complexity, increase the time required to complete the procedure, and potentially even implicate achieving a successful outcome.

Thus, it would be desirable to provide an apparatus that maintains substantially uninterrupted blood flow in one vessel where such is critical, such as the aorta, while isolating the site of a puncture in that vessel as well as an adjacent vessel, such as the renal artery, to facilitate placement of a stent-graft or the like.

### SUMMARY OF THE INVENTION

An object of the disclosure is to provide an apparatus that maintains substantially uninterrupted blood flow in one vessel where such is critical, such as the aorta, while isolating the site of a puncture in the vessel as well as in an adjacent vessel, such as the renal artery, to facilitate placement of a stent-graft or the like for achieving a bypass.

According to one aspect of the disclosure, an apparatus for performing a medical procedure in a main vessel connected to a branch vessel is disclosed. The apparatus comprises a first inflatable balloon forming an internal passage for allowing perfusion in the main vessel when the inflatable balloon is inflated. A shaft includes an inflation lumen in communication with the inflatable balloon, and a working lumen including an outlet for passing or receiving a tool, such as a retractable needle for puncturing the vessel. A second inflatable balloon is connected to the shaft for insertion into the branch vessel when inflated.

In one embodiment, the shaft comprises a flexible elbow extending along an external surface of the first inflatable balloon. The shaft may further comprise a first port for communicating inflation fluid to an interior compartment of the inflatable balloon. The external surface of the first inflatable balloon may be located in a recess for receiving a portion of the shaft. The outlet of the working lumen may be substantially flush with a portion of the external surface of the first inflatable balloon, which may be compliant or semi-compliant.

The second inflatable balloon may comprise an indexing balloon connected to the shaft for expanding into a branch vessel to orient the working lumen in the direction of the branch vessel, such as to allow for an incision to be made at a desired location for achieving a bypass. The shaft further includes a second port in fluid communication with an interior compartment of the indexing balloon. The indexing balloon may be adapted for being at least partially inserted into the branch vessel. The indexing balloon may be positioned proximally of the first inflatable balloon on the shaft, and oriented in substantially a common direction as an outlet of the working lumen.

The shaft may be connected to an external surface of the first inflatable balloon. The shaft may be connected between two layers of the first inflatable balloon.

According to a further aspect of the disclosure, an apparatus for performing a medical procedure in a main vessel connected to a branch vessel is provided. A first inflatable balloon forms an internal passage for allowing perfusion in the main vessel when the first inflatable balloon is inflated. A shaft is connected to the first inflatable balloon, the shaft including an inflation lumen in communication with the first inflatable balloon. A second inflatable balloon is connected to the shaft for insertion into the branch vessel when inflated.

The second inflatable balloon is positioned proximally of the first inflatable balloon along the shaft, and oriented in substantially a common direction as an outlet of a working lumen of the shaft. The second inflatable balloon may be in fluid communication with the inflation lumen of the shaft. The second inflatable balloon may include a projection oriented in a common direction as the outlet of the working lumen.

The shaft may further include a working lumen including an outlet for passing or receiving a retractable needle for puncturing the vessel. The shaft may comprise an elbow, and may include a port for communicating inflation fluid to an interior compartment of the first inflatable balloon. The shaft may be connected to an external surface of the first inflatable balloon, which may be located in a recess for receiving a distal end portion of the shaft. The shaft may also include a working lumen having an outlet substantially flush with the external surface of the balloon. In any version, the first inflatable or perfusion balloon may be compliant or semi-compliant and the second inflatable or indexing balloon may be non-compliant.

According to a further aspect of the disclosure, a method of performing a medical procedure at a junction between a main vessel including a first fluid flow and a branch vessel. The method comprises positioning a first inflatable balloon on a catheter shaft at least partially in the branch vessel. The method further comprises positioning a second inflatable perfusion balloon on the catheter shaft in the main vessel so as to allow the first fluid flow therein when the second inflatable perfusion balloon is inflated.

The method may further include the step of puncturing one of the main or branch vessels. The puncturing step may comprise deploying a needle from the catheter through the catheter shaft.

### BRIEF DESCRIPTION OF THE DRAWINGS

A better understanding of the above and further advantages of the present disclosure results from referring to the following description in conjunction with the accompanying drawings in which:

FIG. 1 is a partially cutaway perspective view of one exemplary embodiment of a perfusion balloon catheter;

FIG. 1A is a cross-sectional view of the embodiment of FIG. 1;

FIG. 1B is a perspective view of the perfusion balloon catheter of FIG. 1;

FIG. 1C is a cross-sectional view of a catheter shaft for the perfusion balloon catheter of FIG. 1;

FIG. 1D is a perspective end view of the catheter of FIG. 1;

FIG. 1E is an isolated view of a perfusion balloon of the catheter of FIG. 1;

FIG. 1F is an isolated view of an indexing balloon of the catheter of FIG. 1;

FIGS. 2, 2A, 2B, and 2C are schematic progressive views of a possible use of a perfusion balloon catheter according to this disclosure; and

FIGS. 3, 3A, 3B, and 3C illustrate an alternate embodiment of a perfusion balloon catheter.

The dimensions of some of the elements may be exaggerated relative to other elements for clarity or several physical components may be included in one functional block or element. Further, sometimes reference numerals may be repeated among the drawings to indicate corresponding or analogous elements. Moreover, some of the parts depicted in the drawings may be combined into a single function.

### DETAILED DESCRIPTION

In the following detailed description, numerous specific details are set forth to provide a thorough understanding of the presently disclosed invention(s). The disclosed embodiments may be practiced without these specific details. In other instances, well-known methods, procedures, components, or structures may not have been described in detail so as not to obscure the disclosed inventive concepts.

The invention of this disclosure is not limited in its application to the details of construction and the arrangement of the components set forth in the following description or illustrated in the drawings. The inventive concepts disclosed are capable of other embodiments or of being practiced or carried out in various ways. It is also to be understood that the phraseology and terminology employed herein are for the purpose of description and should not be regarded as limiting.

Certain features of the disclosed embodiments that are, for clarity, described in the context of separate embodiments, may also be provided in combination in a single embodiment. Conversely, various features of the invention that are, for brevity, described in the context of a single embodiment, may also be provided separately or in any suitable sub-combination.

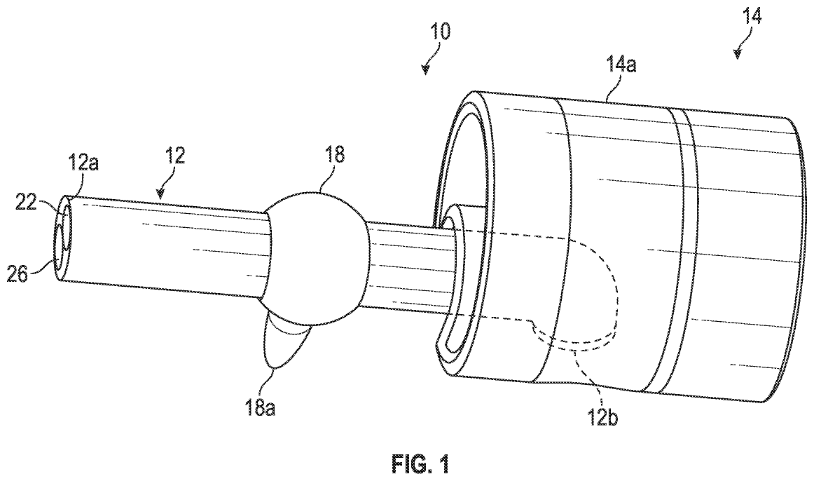

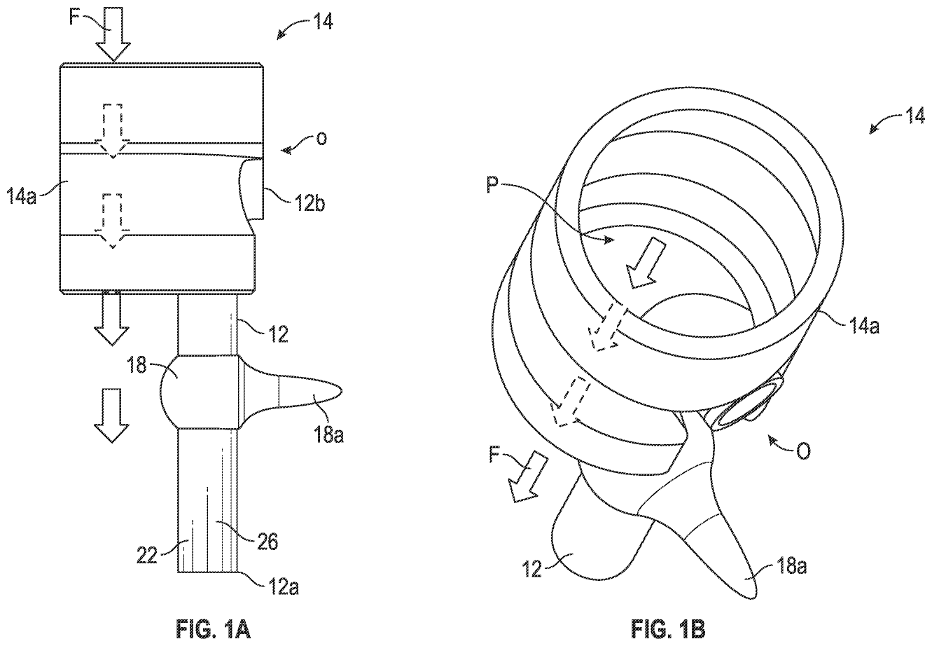

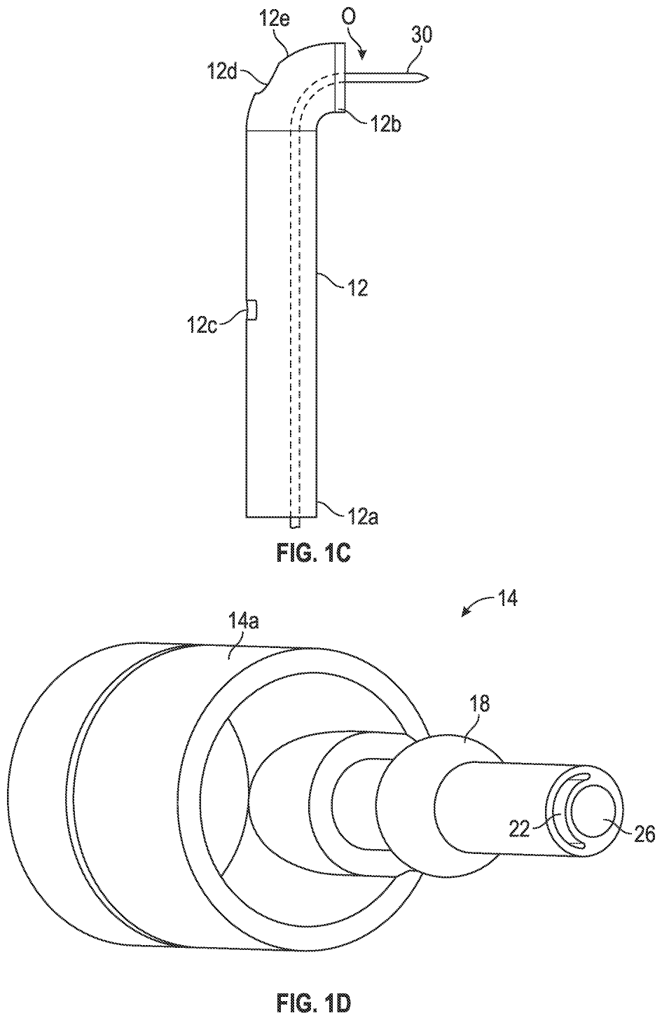

With reference to FIG. 1, a perfusion catheter 10 is provided for endovascular use and, in particular, one that may be adapted for use in selectively puncturing a primary or main vessel, such as for forming a shunt or bypass to a secondary or branch vessel using a stent-graft or the like. In the illustrated example, the perfusion catheter 10 includes a shaft 12 having a proximal end 12a and a distal end 12b (which would normally be spaced apart a substantial distance, such that when the proximal end 12a is external to the body, the distal end 12b is adjacent a site for treatment, but shaft is shown in an abbreviated form for purposes of illustration only).

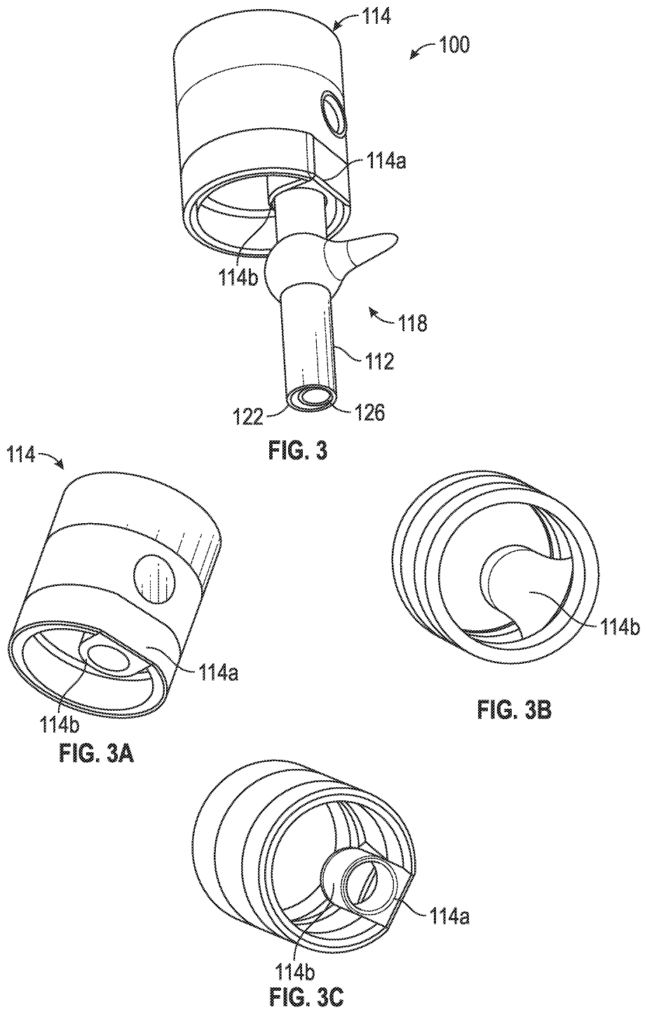

The distal end 12b of the shaft 12 supports an expandable element, which in the illustrated embodiment comprises of an expandable perfusion balloon 14. This perfusion balloon 14 is connected to the shaft 12, such as along an external surface 14a in a manner outlined in the following description. Alternatively, the shaft 12 could be positioned between two layers of the balloon 14, as shown in FIG. 3 and outlined further in the following description. The balloon 14 is generally annular in shape in the inflated condition, as shown in FIGS. 1, 1A, 1B, and 1C, and thus when inflated includes an internal passage P that allows for fluid perfusion (as indicated by action arrow F) along a vessel when deployed therein and inflated.

The balloon 14 may be compliant or semi-compliant, meaning it may conform to the shape of an external structure when inflated, such as the inner wall of a vessel. This helps to ensure the balloon 14 can be expanded to make substantially complete contact with the inner wall of the vessel, the diameter of which may differ among patients. A typical material for forming such a balloon would be an elastomer, such as polyurethane or silicone. However, a non-compliant balloon (which does not further expand beyond a nominal diameter with added fluid pressure) could also be used if sized appropriately to accommodate the vessel. When deflated (such as prior to inflation and during introduction to the vasculature), the balloon 14 may fold or collapse over a distal end 12b of the shaft 12.

The shaft 12 may also support a second inflatable balloon 18, which as described in further detail below may serve to provide a locating or indexing function for the catheter 10 in use (such as by orienting the working lumen in the direction of the branch vessel, such as to allow for an incision to be made at a desired location for achieving a bypass). This indexing balloon 18 is shown positioned proximally of the perfusion balloon 14, and may also be connected to the shaft 12. As can be seen, the indexing balloon 18 has a transversely projecting portion or finger 18a, the function of which will be explained in the following description.

This indexing balloon 18 may be non-compliant, and may be fabricated of inelastic materials, such as nylon or polyester, with or without the addition of fibers. The projecting portion or finger 18a may include an identifier to allow for visualization under fluoroscopy, such as for example a radiopaque marker. The end geometry of the indexing balloon 18 may vary depending on what is needed for the branch vessel and depending on the degree of stenosis.

As can be understood from FIGS. 1A and 1C, the shaft 12 includes a plurality of internal passages or lumens extending from the proximal end 12a to the distal end 12b. These passages include an inflation lumen 22, which may communicate with the balloons 14 and 18. Specifically, the shaft 12 may include a first port 12c along a linear portion in communication with an interior compartment of the indexing balloon 18 connected to this portion of the shaft. As perhaps best shown in FIG. 1C, a second port 12d may be provided along a distal end portion of the shaft 12 including a bend or elbow 12e. This end portion of the shaft 12 may be flexible to facilitate bending for achieving the desired orientation facing the vessel inner wall and to communicate fluid to an interior compartment of the perfusion balloon 14 via the second port 12d.

A working lumen 26 may also be provided within the shaft 12 for receiving a working implement or tool, such as for example a cutter in the form of an elongated puncture needle 30 as shown in FIG. 1C (but other tools could be adapted for one or more of impedance sensing, graft delivery, or laser ablation to seal the vessel walls to the graft). When advanced a suitable distance, such as by pushing a proximal end, a distal end of this needle 30 may alight through an opening or outlet O of the working lumen 26 to puncture a vessel wall, and then retract into the working lumen when the operation is completed. The opening or outlet O may be in a transverse direction, along a lateral side of the shaft 12. The location of the opening or outlet O in the shaft 12 may include an identifier to allow for visualization under fluoroscopy, such as for example a radiopaque marker. A suitable hub (not shown) may also be provided at the proximal end 12a of the shaft 12 for communicating with each lumen 22, 26.

FIG. 1E illustrates the perfusion balloon 14 isolated from the catheter 10 and, in particular shaft 12. It can be understood that this generally annular balloon 14 includes a recess 14b including a portion of the external surface 14a. This recess 14b may be generally U-shaped with an open upper end and have a length less than that of the balloon 14, such that the curved portion, or elbow 12e of the shaft 12 may be located therein. This would be optional if the alternative of having the shaft 12 between the two layers of the balloon 14, as outlined below in more detail, is used.

As can be understood from FIG. 1B, the dimension of the recess 14b may be such that the distal end 12b of the shaft 12 and, in particular, the opening or outlet O of the working lumen 26, is substantially flush with the adjacent portion of the external surface 14a of the balloon 14. Yet, the extension of the recess 14b radially inward is not sufficiently great to meaningfully interfere with fluid perfusion, as can be understood from FIGS. 1A and 1B.

In this manner, when the balloon is advanced into engagement with an external structure, such as a vessel inner wall, the outlet O is positioned in close proximity to the corresponding surface. Concurrently, the inflation of the balloon 14 tends to prevent fluid flow at this location (but not perfusion through the balloon 14 and hence the main or primary vessel). When the wall is thus punctured by an implement, such as needle 30, loss of fluid is thus minimized or prevented.

Turning to FIG. 1F, the second or indexing balloon 18 is shown isolated from the catheter 10 and shaft 12. As can be understood, the projecting portion or finger 18a may be tapered in a transverse direction, and thus adapted to be at least partially inserted into a side or branch vessel when the catheter 10 is located in a primary vessel. The indexing balloon 18 also includes an internal passage 18b for receiving the shaft 12, oriented such that the finger 18a extends in the same direction as the outlet O of the working lumen 26, thus aiding the clinician in ensuring the proper alignment results for achieving successful puncture.

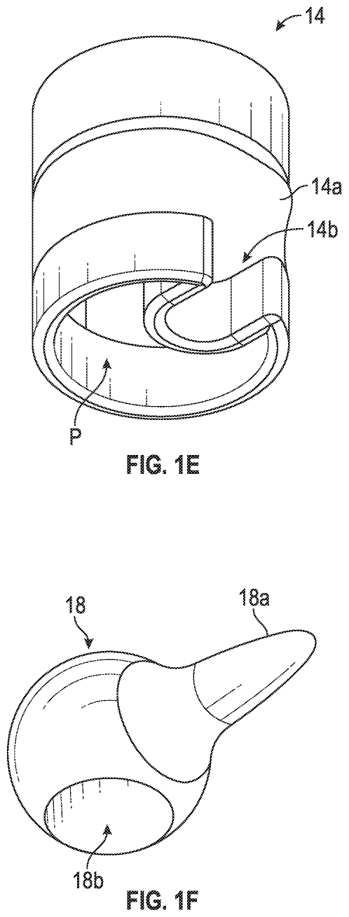

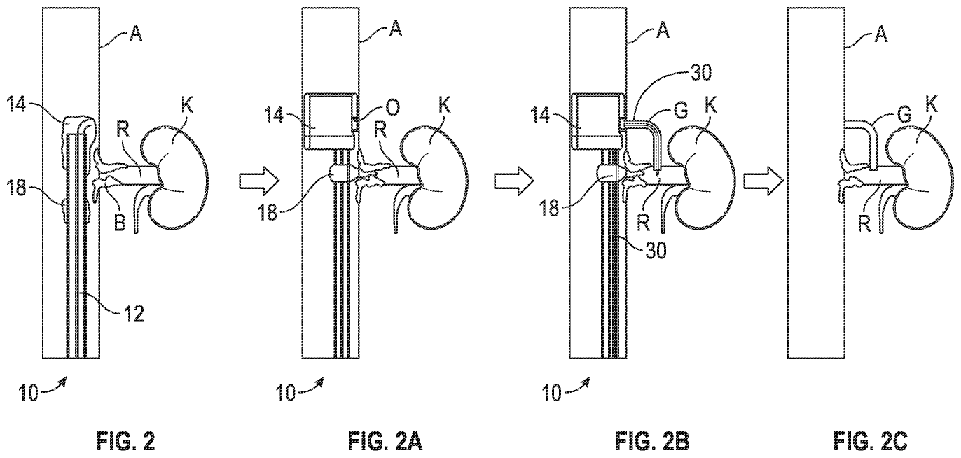

In use, and with reference to FIG. 2, the catheter 10 may be inserted into a primary vessel, such as the aorta A, from a remote location (such as the iliac artery) via guidance provided by a previously installed guidewire (not shown) passing though working lumen 26 and a suitable sheath (not shown). Using fluoroscopy (to identify the location of one or more radiopaque markings on the shaft 12, as noted below), the catheter 10 may be advanced to a location of a branch vessel, such as the renal artery R, where a shunt is desired, such as because of a blockage B occluding blood flow to a kidney K. The positioning is such that the balloon 14 is located distally of the branch vessel R, and the indexing balloon 18 is located adjacent to the branch vessel R, as shown in FIG. 2.

The balloons 14, 18 may then be inflated simultaneously via inflation lumen 22 from a remote inflation source, such as an indeflator (not shown) connected to a hub at a portion of the proximal end 12a of the shaft 12 external to the patient. Consequently, the balloon 14 expands and engages the inner wall of the main vessel (e.g., aorta A), as illustrated. Despite this expansion, the open annular nature of the balloon 14 allows blood flow to perfuse, including through an internal passage of the inflated balloon 14, while the compliant nature allows for it to seal reliably against the vessel wall (despite the fact that different vessel diameters will be present among different patients).

As can be appreciated, when balloon 18 is inflated, it extends into the branch vessel (e.g., renal artery R), and transverse or cross fluid flow therein is substantially occluded. This extension also helps to assure the proper positioning and orientation of the outlet O of the working lumen 26 along the vessel inner wall to allow for puncturing at a suitable location, such as to create the desired bypass.

The needle 30 may be advanced through the working lumen 26, until the tip thereof extends out through the corresponding opening or outlet O of the working lumen 26 of the shaft 12. The puncture site is advantageously substantially sealed against leakage or extravasation by the inflation of the compliant or semi-compliant balloon 14 in the main vessel (e.g., aorta A). The needle 30 may be passed into engagement with the branch vessel, such as renal artery R, to puncture the same. In both cases, the puncturing operation may be facilitated by forming the needle 30 of a shape memory material, such as Nitinol, which may induce a curvature to allow for the needle 30 to enter and puncture the branch vessel in a preferential manner.

Once the puncture is completed, the needle 30 may be retracted into the shaft 12. The desired operation may be completed, such as in the case of a bypass by inserting a graft G through the working lumen 26. This graft G may be attached in place to allow an alternative pathway for fluid communication among the main and branch vessels, and avoid the occluded portion of the branch vessel.

When the procedure is completed, the balloons 14, 18 may be deflated and refolded. This may involve using the previously introduced sheath (not shown). The catheter 10 may then be withdrawn from the vessel, or positioned at another location for further use in performing a procedure.

FIGS. 3, 3A, 3B, and 3C illustrate an alternative version of a catheter 100 including a perfusion balloon 114 in which the shaft 112 is located between layers of material 114a, 114b forming the balloon. These layers 114a, 114b may thus form a receiver with an opening for passing a distal end portion of the shaft 112, which as shown in FIG. 3 may be flexible so as to form an elbow. In this manner, the outlet of the working lumen 126 is substantially flush with the balloon outer surface. The inflation lumen 122 may be used to inflate the perfusion balloon 114, as well as the indexing balloon 118, in the manner previously described.

Summarizing, this disclosure may be considered to relate to the following items:

1. An apparatus for performing a medical procedure in a main vessel connected to a branch vessel, comprising:
a first inflatable balloon forming an internal passage for allowing perfusion in the main vessel when the inflatable balloon is inflated and having an external surface;
a shaft including an inflation lumen in communication with the inflatable balloon, and a working lumen including an outlet for passing a tool, such as a retractable needle for puncturing the vessel; and
a second inflatable balloon connected to the shaft for insertion into the branch vessel when inflated.

2. The apparatus of item 1, wherein the shaft comprises an elbow extending along the external surface of the first inflatable balloon.

3. The apparatus of item 1 or item 2, wherein the shaft comprises a first port for communicating inflation fluid to an interior compartment of the first inflatable balloon.

4. The apparatus of any of items 1-3, wherein the external surface of the first inflatable balloon is located in a recess for receiving a portion of the shaft.

5. The apparatus of any of items 1-4, wherein the outlet of the working lumen is substantially flush with a portion of the external surface of the first inflatable balloon.

6. The apparatus of any of items 1-5, wherein the first inflatable balloon is compliant or semi-compliant.

7. The apparatus of any of items 1-6, wherein the second inflatable balloon comprises an indexing balloon connected to the shaft, the shaft further including a second port in fluid communication with an interior compartment of the indexing balloon.

8. The apparatus of item 7, wherein the indexing balloon is adapted for being at least partially inserted into the branch vessel.

9. The apparatus of item 7 or item 8, wherein the indexing balloon is positioned proximally of the first inflatable balloon on the shaft, and oriented in substantially a common direction as an outlet of the working lumen.

10. The apparatus of any of items 1-9, wherein the shaft is connected to an external surface of the first inflatable balloon.

11. The apparatus of any of items 1-9, wherein the shaft is connected between two layers of the first inflatable balloon.

12. An apparatus for performing a medical procedure in a main vessel connected to a branch vessel, comprising:
a first inflatable balloon forming an internal passage for allowing perfusion in the main vessel when the first inflatable balloon is inflated;
a shaft connected to the first inflatable balloon, the shaft including an inflation lumen in communication with the first inflatable balloon; and
a second inflatable balloon connected to the shaft for insertion into the branch vessel when inflated.

13. The apparatus of item 12, wherein the second inflatable balloon is positioned proximally of the first inflatable balloon along the shaft, and oriented in substantially a common direction as an outlet of a working lumen of the shaft.

14. The apparatus of item 12 or item 13, wherein the second inflatable balloon is in fluid communication with the inflation lumen of the shaft.

15. The apparatus of any of items 12-14, wherein the shaft further includes a working lumen including an outlet for receiving a retractable needle for puncturing the vessel.

16. The apparatus of any of items 12-15, wherein the shaft comprises an elbow.

17. The apparatus of any of items 12-16, wherein the shaft comprises a port for communicating inflation fluid to an interior compartment of the first inflatable balloon.

18. The apparatus of any of items 12-17, wherein the shaft is connected to a portion of an external surface of the first inflatable balloon.

19. The apparatus of any of items 10-18, wherein the shaft is connected between two layers of the first inflatable balloon.

20. The apparatus of item 18, wherein a portion of the external surface of the first inflatable balloon is located in a recess for receiving a distal end portion of the shaft.

21. The apparatus of item 18, wherein the shaft includes a working lumen having an outlet substantially flush with the external surface of the balloon.

22. The apparatus of any of items 12-21, wherein the second inflatable balloon includes a projection oriented in a common direction as the outlet of the working lumen.

23. The apparatus of any of items 12-22, wherein the first inflatable balloon is compliant or semi-compliant and the second inflatable balloon is non-compliant.

24. A method of performing a medical procedure at a junction between a main vessel including a first fluid flow and a branch vessel, comprising:
positioning a first inflatable balloon on a catheter shaft at least partially in the branch vessel; and
positioning a second inflatable perfusion balloon on the catheter shaft in the main vessel so as to allow the first fluid flow therein when the second inflatable perfusion balloon is inflated.

25. The method of item 24, further including the step of puncturing one of the main or branch vessels.

26. The method of item 24, wherein the puncturing step comprises deploying a needle from the catheter through the catheter shaft.

As used herein, the following terms have the following meanings:

“A”, “an”, and “the” as used herein refers to both singular and plural referents unless the context clearly dictates otherwise. By way of example, “a compartment” refers to one or more than one compartment.

“About,” “substantially,” “approximately,” or “generally” as used herein referring to a measurable value, such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosed invention. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows, e.g., component or the like does not exclude or preclude the presence of additional, non-recited components, features, element, members, steps, known in the art or disclosed therein.

Although the invention is described in conjunction with specific embodiments, many alternatives, modifications, and variations will be apparent to those skilled in the art. Accordingly, this disclosure embraces all such alternatives, modifications, and variations that fall within the spirit and scope of the appended claims. It is to be fully understood that certain aspects, characteristics, and features, of the invention, which are, for clarity, illustratively described and presented in the context or format of a plurality of separate embodiments, may also be illustratively described and presented in any suitable combination or sub-combination in the context or format of a single embodiment. Conversely, various aspects, characteristics, and features, of the invention which are illustratively described and presented in combination or sub-combination in the context or format of a single embodiment may also be illustratively described and presented in the context or format of a plurality of separate embodiments.

1. An apparatus for performing a medical procedure in a main vessel connected to a branch vessel, comprising:
a first inflatable balloon forming an internal passage for allowing perfusion in the main vessel when the inflatable balloon is inflated, the balloon having an external surface;
a shaft including an inflation lumen in communication with the inflatable balloon, and a working lumen including an outlet along the external surface for passing a tool; and
a second inflatable balloon connected to the shaft for insertion into the branch vessel when inflated.

2. The apparatus of claim 1, wherein the shaft comprises an elbow extending along a portion of the external surface of the first inflatable balloon.

3. The apparatus of claim 1, wherein the shaft comprises a first port for communicating inflation fluid to an interior compartment of the first inflatable balloon.

4. The apparatus of claim 1, wherein a portion of the external surface of the first inflatable balloon is located in a recess for receiving a portion of the shaft.

5. The apparatus of claim 1, wherein the outlet of the working lumen is substantially flush with a portion of the external surface of the first inflatable balloon.

6. (canceled)

7. The apparatus of claim 1, wherein the second inflatable balloon comprises an indexing balloon connected to the shaft, the shaft further including a second port in fluid communication with an interior compartment of the indexing balloon, wherein the indexing balloon is adapted for being at least partially inserted into the branch vessel, or the indexing balloon is positioned proximally of the first inflatable balloon on the shaft, and includes a transversely projecting portion oriented in substantially a common direction as an outlet of the working lumen.

8.-9. (canceled)

10. The apparatus of claim 1, wherein the shaft is connected to the external surface of the first inflatable balloon.

11. The apparatus of claim 1, wherein the shaft is connected between two layers of the first inflatable balloon.

12. An apparatus for performing a medical procedure in a main vessel connected to a branch vessel, comprising:
a first inflatable balloon forming an internal passage for allowing perfusion in the main vessel when the first inflatable balloon is inflated;
a shaft connected to the first inflatable balloon, the shaft including an inflation lumen in communication with the first inflatable balloon; and
a second inflatable balloon connected to the shaft for insertion into the branch vessel when inflated.

13. The apparatus of claim 12, wherein the second inflatable balloon is positioned proximally of the first inflatable balloon along the shaft, and oriented in substantially a common direction as an outlet of a working lumen of the shaft.

14. The apparatus of claim 12, wherein the second inflatable balloon is in fluid communication with the inflation lumen of the shaft, and the shaft further includes a working lumen including an outlet for receiving a retractable needle for puncturing the main or branch vessels.

15. (canceled)

16. The apparatus of claim 12, wherein the shaft comprises an elbow.

17. The apparatus of claim 12, wherein the shaft comprises a port for communicating inflation fluid to an interior compartment of the first inflatable balloon.

18. The apparatus of claim 12, wherein the shaft is connected to a portion of an external surface of or between two layers of the first inflatable balloon.

19. The apparatus of claim 18, wherein the portion of the external surface of the first inflatable balloon is located in a recess for receiving a distal end portion of the shaft.

20. The apparatus of claim 18, wherein the shaft includes a working lumen having an outlet substantially flush with the external surface of the balloon.

21. The apparatus of claim 20, wherein the second inflatable balloon includes a projection oriented in a common direction as the outlet of the working lumen.

22. The apparatus of claim 12, wherein the first inflatable balloon is compliant or semi-compliant and the second inflatable balloon is non-compliant.

23. A method of performing a medical procedure at a junction between a main vessel including a first fluid flow and a branch vessel, comprising:
positioning a first inflatable balloon on a catheter shaft at least partially in the branch vessel; and
positioning a second inflatable perfusion balloon on the catheter shaft in the main vessel so as to allow the first fluid flow therein when the second inflatable perfusion balloon is inflated.

24. The method of claim 23, further including the step of puncturing one of the main or branch vessels by deploying a needle from the catheter through the catheter shaft.

25. (canceled)
